(19) United States
(12) Patent Application Publication
(10) Pub. No.: US 2011/0092830 A1
(43) Pub. Date: Apr. 21, 2011

(54) METHOD FOR AUTOMATIC ERROR DETECTION IN PRESSURE MEASUREMENT AND AN ELECTRONIC SPHYGMOMANOMETER

(75) Inventors: Yunquan Chen, Delta (CA); Yun Xu, Beijing (CN)

(73) Assignee: PRECISION MEDICAL TECHNOLOGY LTD., Beijing (CN)

(21) Appl. No.: 12/994,903

(22) PCT Filed: May 27, 2009
(86) PCT No.: PCT/CN09/72031

§ 371 Date: Nov. 29, 2010

(30) Foreign Application Priority Data
May 27, 2008 (CN) 200810113188.5

(51) Int. Cl. A61B 5/022; G06F 19/00; G01L 27/00

(52) U.S. Cl. 600/490; 702/98

(57) ABSTRACT

This invention discloses a method for automatic error detection in pressure measurement and an electronic sphygmomanometer using the method. The electronic sphygmomanometer comprises two pressure sensing circuits connected to a MPU. The first pressure sensing circuit is normally-on in pressure measurement. The second pressure sensing circuit is normally-off in pressure measurement. Said normally-off pressure sensing circuit is periodically turned on in an automatic manner to measure the same pressure as the normally-on pressure sensing circuit is used to measure. Said MPU computes the difference between the pressures obtained from the two pressure sensing circuits. When the difference is greater than a given error limit, calibration error warning will be given.

### TECHNICAL FIELD

This invention is related to a pressure measurement method and an electronic sphygmomanometer. In particular, this invention is related to a method for automatic error detection in pressure measurement and electronic sphygmomanometer which uses said error detection method.

### BACKGROUND

Electronic sphygmomanometers comprise pressure sensing circuits for measurement of air pressure in an inflatable cuff applied to occlude the artery of the subject. The measurement of blood pressure may be automatic using the oscillometric method or manual using a stethoscope to listen to the Korotkoff sounds by the operator.

The pressure sensor circuits used for measurement of pressure are typically calibrated in manufacturing. However, the pressure sensing circuits may lose their calibration in use due to numerous factors including usage time, environmental impact, aging of electronic components, temperature changes, failure of material, etc.

To ensure the sensor circuits meet accuracy requirement, they are typically required to be calibrated regularly by a skilled person. However, regular calibration may be inconvenient to users and may increase the cost of using them.

### SUMMARY OF INVENTION

This invention provides an automatic error detection method and an electronic sphygmomanometer which uses this method. Said automatic error detection method is implemented with the combination of electronic hardware and software programs. The electronic hardware includes a normally-on pressure measurement channel and a normally-off pressure measurement channel under, the control of a micro-processor or micro-controller unit (MPU). The normally-on pressure measurement channel comprises a pressure sensor and an electronic circuit commonly used in pressure measurement. The normally-off pressure measurement channel also comprises a pressure sensor and an electronic circuit, but they are normally turned off during use unless being turned on for calibration.

When the normally-on pressure measurement channel has been used for a certain amount of time or a certain number of times, the normally-off pressure measurement channel shall automatically starts pressure measurement under the control of the MPU to do error detection for the normally-on pressure measurement channel. Software programs implemented in said MPU include accumulating and recording the time of use or the number of usage times of the normally-on pressure measurement channel, controlling a hardware switch of the normally-off pressure measurement channel, using the normally-on and normally-off pressure measurement channels to measure the same input pressure at the same time and calculating the difference of the two measured pressures, determining whether the normally-on or normally-off channel has lost calibration, and displaying the result of automatic error detection.

Said normally-off pressure measurement channel and automatic error detection may be started daily, weekly or monthly, or every 2, 5, 10, 20 or 50 usage times of the normally-on pressure measurement channel.

The automatic error detection method in pressure measurement provided by this invention may be applied to all types of electronic sphygmomanometers, including manual or automatic electronic sphygmomanometer measuring blood pressure by either the Korotkoff sound method or the oscillometric method.

The power supply to the second pressure measurement channel is under the control of the MPU. This may be achieved by an electronic hardware switch which is independent of the MPU. It may also be achieved by an I/O port of the MPU controlled by the software in the MPU. The second pressure sensor may be connected to the inflatable part by a switchable valve under the control of the MPU. The MPU selectively pressurizes the second pressure sensor via this switchable valve.

Further aspects of the invention and features of specific embodiments of the invention are described below.

### BRIEF DESCRIPTION OF THE DRAWINGS

In drawings which illustrate non-limiting embodiments of the invention,

FIG. 1 is a block diagram of the electronic hardware of an electronic sphygmomanometer with automatic error detection;

FIG. 2 is a software program flowchart of an electronic sphygmomanometer with automatic error detection.

The marks of the drawings are as follows:

22—inflation part; 24—inflatable part; 25—deflation valve; 26—the first channel pressure sensor; 27—electromagnetic valve; 28—the second channel pressure sensor; 30—the first channel differential amplifier; 32—the second channel differential amplifier; 34—MPU; 36—display; 52—initialization; 54—error record determination; 56—calibration warning; 57—measurement time recording; 58—measurement time determination; 60—electromagnetic valve and the second pressure measurement channel power-on switching; 62—data acquisition; 63—inflation period determination; 64—pressure display updating; 65—error detection determination; 66—deflation rate determination; 67—absolute difference calculation; 68—error determination; 70—absolute difference recording; 72—blood pressure measurement; 74—end of blood pressure measurement determination; 76—blood pressure measurement result display; 78—error record determination; 80—calibration warning; 82—program end.

### DETAILED DESCRIPTION OF EMBODIMENT

Embodiment of the automatic error detection method and the electronic sphygmomanometer using said method will be described by using the same description of the embodiment of an electronic sphygmomanometer.

Throughout the following description, specific details are set forth in order to provide a more thorough understanding of the invention. However, the invention may be practiced without these particulars. In some cases, well known elements have not been shown or described in detail to avoid unnecessarily obscuring the invention. Accordingly, the specification and drawings are to be regarded in an illustrative, rather than a restrictive, sense.

An electronic sphygmomanometer comprises an inflation part, a deflation part, an inflatable part connected with the inflation and deflation parts, a first channel pressure sensor and electronic circuit connected with the inflatable part for pressure measurement, a MPU connected with the first channel pressure sensor and electronic circuit, a display under the control of the MPU, and a second channel pressure sensor and electronic circuit for automatic error detection which is in, parallel with the first channel pressure sensor and electronic circuits. The first channel pressure sensor and electronic circuit is a normally-on pressure measurement channel of the electronic sphygmomanometer. The second channel pressure sensor and electronic circuit is a normally-off pressure measurement channel of the electronic sphygmomanometer.

The power of the second channel pressure sensor and electronic circuit is supplied through a detection power switch which is under the control of the MPU. This switch may be an electronic hardware switch independent of the MPU, or it may also be a “soft” switch using an I/O port of the MPU controlled by software embedded in the MPU. The second channel pressure sensor and its electronic circuits may be selectively powered through this switch. The input of the second pressure sensor may be further connected with the inflatable part through a switchable valve under the control of the MPU; the MPU selectively pressurizes the second channel pressure sensor through the switchable valve.

Said detection power switch is normally off, thus the second channel pressure sensor and its electronic circuit are normally not powered. Similarly, said switchable valve is normally off, thus the input pressure is to the second channel pressure sensor is normally zero. Every time the operator uses the electronic sphygmomanometer, the pressure measurement is done by the first channel pressure sensor and its electronic circuit, and every time at the end of the blood pressure measurement, the MPU will update the number of usage times record of the electronic sphygmomanometer.

There is an initialization process every time said electronic sphygmomanometer is powered on, the initialization process includes checking the number of usage times record of the sphygmomanometer. When this number of usage times is a multiple of a predetermined number, the MPU will turn on the switchable valve and detection power switch to start the second channel pressure sensors and its electronic circuits, and do automatic error detection for the first channel pressure sensor and its electronic circuit. Said predetermined times are at least 2. It may also be 5, 10, 20 or 50.

When the number of usage times is a multiple of a predetermined number, said electronic sphygmomanometer measures the pressure in the inflatable part using both the two independent pressure sensors and electronic circuits at the same time, and input the two generated pressure signals to the MPU which calculates the error between them, and displays this error on the display. If the error is greater than a given allowed value, for example, 4 mmHg or 2% of pressure readings (take the greater of the two), the error on display may be flashed for warning to the operator. This error detection may be done automatically during the blood pressure measurement.

In the process of error detection, if an error is detected to be greater than a given allowable value, it is likely that the sphygmomanometer has lost calibration, and needs re-calibration. At this time the MPU will record the absolute value of this error. The MPU only records the maximum absolute error value that is greater than the given allowable value. If there has already been error recorded in the MPU at start up, then the absolute error value will be displayed before the next blood pressure measurement is started.

As a result of two independent measurement systems measuring the same pressure in the inflatable part at the same time, the response time for the hardware of the two systems may be different; and there may be a time delay for the pressure to reach the two systems when measuring dynamic pressure, which results in time difference, so that the measured pressure by the two systems may be different at the same time, likely generating measurement error during error detection. Therefore, measurement error may be reduced if we do the error measurement when the rate of pressure change in the inflatable part is small. So the automatic error detection is better set up to be done in the slow deflation period after the inflation period in blood pressure measurement.

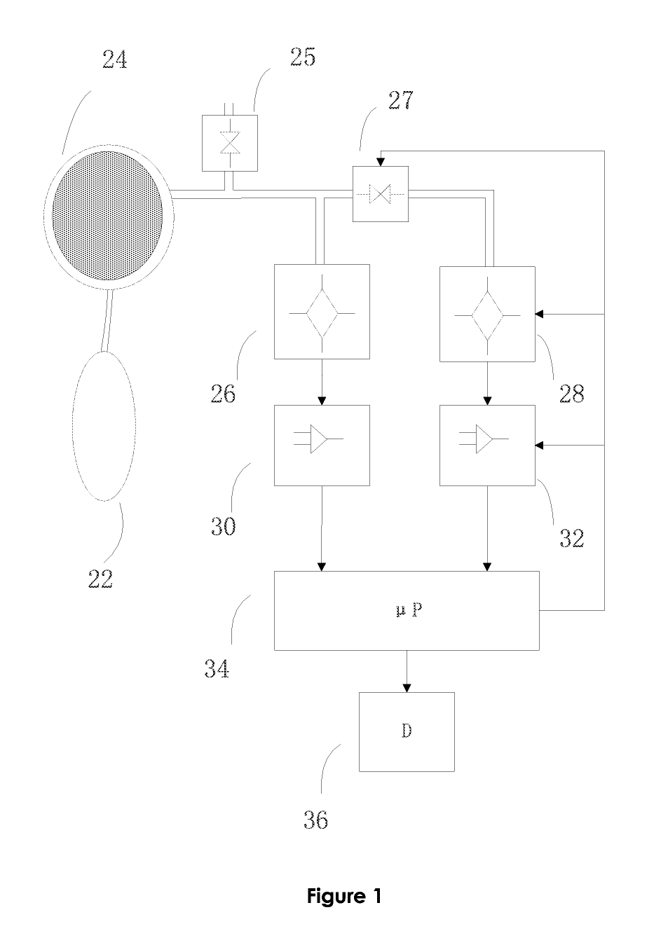

As shown in FIG. 1, an electronic sphygmomanometer with automatic error detection in pressure measurement comprises inflation part 22, inflatable part 24, deflation valve 25, first channel pressure sensor 26, second channel pressure sensor 28, first differential amplifier 30, second channel differential amplifier 32, MPU 34, and display 36. The inflation part 22 may be a manually pump; it may also be electric air pump. The inflatable part may be an arm cuff, or wrist cuff. Deflation valve 25 may be a manual or automatic deflation valve. The first channel pressure sensor 26 and the first channel differential amplifier 30 may be separate parts or an integrated part. Similarly, the second channel pressure sensor 28 and the second channel differential amplifier 32 may be separate parts or an integrated part. Display 36 may be an LCD or digital LED or graphics display. Said electronic sphygmomanometer may further comprise electromagnetic valve 27. Electromagnetic valve 27 is controlled by MPU 34, and turned on and off by a drive current.

As shown in FIG. 1, when said electronic sphygmomanometer begins to measure pressure, the pressure in the inflatable part 24 is measured by the first channel pressure sensor 26, the pressure signal generated by the first channel pressure sensor 26 is differentially amplified by the first channel differential amplifier 30, and then it is output to the MPU 34, which includes signal acquisition (A/D conversion), processing, and control functions, the MPU 34 will do calculation and processing after recording the pressure signal, and will display the results on display 36. Said electronic sphygmomanometer may automatically measure blood pressure by commonly used oscillometric method, and then displays the measurement results. It may also only display the pressure and allow the operator to do the pressure measurement using the Korotkoff sound method.

In the use of said electronic sphygmomanometer, normally the MPU 36 cuts off the power to the second channel pressure sensor 28 and the second channel differential amplifier 32 through an I/O port, so that the second channel is not in use. If the electromagnetic valve 27 is also used, the electromagnetic valve 27 is normally off, so that normally the pressure in the inflatable part 24 may not flow into the second channel pressure sensor 28.

Every time when the use of the said electronic sphygmomanometer reaches a certain period of time or a certain number of times, the MPU 34 supplies the power for the second channel pressure sensor 28 and the second channel differential amplifier 32 through an I/O port, and does the pressure measurement error detection. If the electromagnetic valve 27 is also used, the electromagnetic valve 27 is also turned on so that the pressure in the inflatable part 24 may be enter into the pressure input port of the second channel pressure sensor 28. As is shown in FIG. 1, when we measure blood pressure with a commonly used method with the first channel pressure sensor 26 and the first channel differential amplifier 30, the second channel pressure sensor 28 and the first channel pressure sensor 26 will measure the same pressure in the inflatable part 24 at the same time. The pressure in the inflatable cuff 24 generates pressure signal through the first channel pressure sensor 26. The signal is amplified by the first channel differential amplifier 30 and sampled by the MPU 34. At the same time, the MPU 34 will also record the pressure that the second channel pressure sensor 28 measured. The MPU 34 will compare and calculate the measured pressure values at the same time between the first channel pressure sensor 26 and the second channel pressure sensor 28, and calculate the absolute value of the difference, that is, the absolute difference (or error value).

For example, for every 10 times that said electronic sphygmomanometer has been used, one pressure measurement error detection may be done. Every time when said electronic sphygmomanometer is powered on, the MPU 34 may read the number of usage times of the said electronic sphygmomanometer from an internal memory, and then increase the number by one and save the number back to said internal memory. Therefore every usage of said electronic sphygmomanometer will be recorded. The MPU 34 determines the number of usage times after recording it. If the number is a multiple of 10, then the MPU 34 will turn on the power 32 of the electromagnetic valve 27, the second channel pressure sensor 28 and the second channel differential amplifier, and start to do automatic error detection during the pressure measurement. If the number of times of this measurement is not a multiple of 10, error detection will not be done.

The MPU 34 will compare the values between the calculated absolute difference and a given allowed error value, the said allowed error may be 4 mmHg or 2% of pressure readings (taking the greater of the two). If said pressure absolute difference value is greater than the given allowed error value, the absolute difference will be recorded. If more than one absolute difference is greater than the given allowed error value, the MPU 34 will record the maximum absolute difference.

In the deflation period of said electronic sphygmomanometer, when the pressure in the inflatable cuff 24 drops to near zero, for example, 5 mmHg or below, it is determined to be the end of the measurement. If there is absolute difference recorded in the MPU 34 after the blood pressure measurement, it indicates the electronic sphygmomanometer has lost calibration. Then the MPU 34 will control display 36 to warn the loss of calibration at the end the blood pressure measurement. On the other hand, before the next blood pressure measurement is started, warning of loss of calibration is also displayed. The way of warning may be to display the absolute difference in an intermittent or flashing display to remind the operator. Other devices, such as a buzzer or red LED indicator light, may be used to remind the loss of calibration of the sphygmomanometer . . . .

In order to reduce measurement error caused by a large rate of pressure change in inflatable cuff 24, resulting in false alarm of loss of calibration of sphygmomanometer, the measurement and calculation of the absolute difference between the pressure values obtained at the same time from the first channel pressure sensor 26 and the second channel pressure sensor 28 may be carried out under the condition that the are of pressure change is small. For example, if the rate of pressure change in the inflatable cuff 24 is over a given rate, then the absolute difference measured is considered invalid. Said given rate may be a pressure decrease of between 5 mmHg and 10 mmHg per second. Since the rate of pressure change in inflation period in blood pressure measurement is greater than that in deflation period, the determination of the absolute difference may be limited in the deflation period.

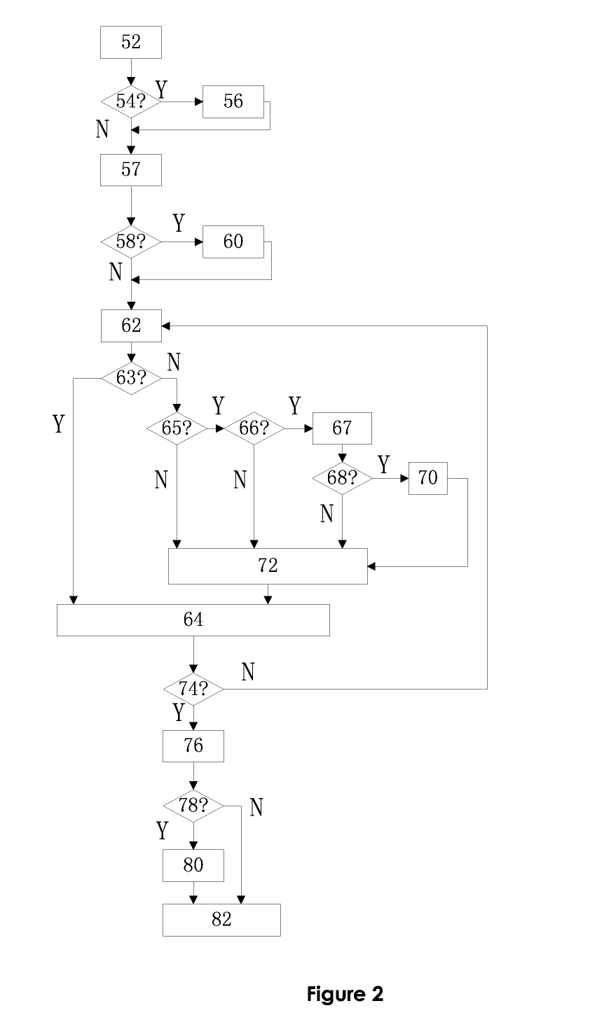

As is shown in FIG. 2, the software program flowchart for automatic error detection in pressure measurement of the electronic sphygmomanometer may include the following steps:
a) Initialization 52 comprises updating the displayed value in displays 34 shown in FIG. 1 and recording the time of updating the display 34. These initial values are zero in general.
b) Error record determination 54 determines whether there is absolute difference recorded in the MPU 34. If yes, it indicates that the sphygmomanometer has lost calibration. If not, the program goes to step d).
c) Calibration warning 56: the MPU 34 controls the display 36 to warn that the electronic sphygmomanometer has lost calibration. Warning may be displayed by intermittent or flashing display of the absolute difference at a rate of about once per second. Total display time may be 5-10 seconds.
d) measurement time recording 57: the MPU 34 records a digital “0” of measurement times at the end of the manufacture of the electronic sphygmomanometer, and then increases the value by one in every initialization in use (to record the measurement times of the current usage times of the sphygmomanometer).
e) Measurement time determination 58 determines whether the measurement time is a multiple of 10. If it is not, the sphygmomanometer will not automatically detect error, and program jumps to step g), the pressure is measured by the first channel pressure sensor 26 only.
f) Electromagnetic valve and the second pressure measurement channel power-on switching 60: the MPU 34 controls the power switch to turn on electromagnetic valve 25 and the second channel pressure sensor 28 and the second channel differential amplifier 32, so as to detect errors for the electronic sphygmomanometer while measuring blood pressure.
g) data acquisition 62 comprises acquiring the pressure data P1 (t) and P2 (t) at current time t in the inflatable cuff 24 shown in FIG. 1, respectively, for the first channel pressure sensor 26 and the second channel pressure sensor 28.
h) Inflation period determination 63 compares the current pressure P1 (t) with the pressure P1 (t−ΔT), where ΔT is between 0.5 to 1.5 seconds, preferably 1 second. If the P1 (t) is not greater than P1 (t−ΔT) by a given pressure value, then the inflation is determined to have ended, the program will jump into the steps k); if P1 (t) is greater than the P1 (t−ΔT) by the given pressure value, the pressure in the inflatable cuff 24 is determined to be in inflation period, the program continues to steps g) to i). Said given pressure value may be a value between 5 mmHg and 10 mmHg
i) Pressure display updating 64: displays on the display 34 shown in FIG. 1 the updated pressure data P1 (t) that the MPU 34 acquired.
j) Repeat steps g) to i) until step h) has determined that the inflation has ended.
k) Error detection determination 65 determines whether the measurement time is a multiple of 10. If the measurement time is not a multiple of 10, then the sphygmomanometer will not automatically detect errors, the program jumps to step p).
l) Deflation rate determination 66 determines whether the deflation rate in inflatable cuff 24 is smaller than a given deflation rated. If the deflation rate in inflatable cuff 24 is greater than a given deflation rate, the absolute difference measurement will not be done, and the program jumps to step p). Said given deflation rate may be between 5 mmHg and 10 mmHg per second.
m) Absolute difference calculation 67 calculates the absolute difference between pressure value P1 (t) from the first channel pressure sensor 26 and the pressure value P2 (t) from the second pressure sensor 28, that is |P1 (t)−P2 (t)|.
n) Error determination 68 determines whether any absolute difference between a set of pressure values is greater than a given allowed error. If the answer is yes, it indicates that the electronic sphygmomanometer has lost calibration. Said given allowed error may be 4 mmHg or 2% of pressure reading (take the greater of the two). If the sphygmomanometer has not lost calibration, the program jumps to step p)
o) Absolute difference recording 70: the MPU 34 records the absolute difference. If more than one set of readings' absolute differences are greater than the given allowed error, the MPU 34 will record the maximum absolute difference.
p) Blood pressure measurement 72 uses the commonly used methods to measure blood pressure including the oscillometric method and Korotkoff sound method (blood pressure measurement methods are known to people in the trade, and shall not be described here).
q) pressure display updating 64 displays on the display 34 shown in FIG. 1 the updated pressure data P1 (t) that the MPU 34 acquired
r) End of blood pressure measurement determination 74: When the pressure in the inflatable cuff 24 has dropped to below 5 mmHg, the pressure measurement is determined to be ended. The information provided in the blood pressure measurement 74 may also be used to determine whether the blood pressure measurement is ended. If blood pressure measurement is not ended, the program repeats steps g) to r) until the measurement is determined to be ended in step r).
s) Blood pressure measurement result display 76: If there is output in blood pressure measurement 72 that needs to be displayed, the MPU 34 will display the results.
t) Error record determination 78 determines whether there is absolute difference recorded in the MPU 34. If none, the program jumps to step v).
u) Calibration warning 80: the MPU 34 controls the display 36 to warn that the electronic sphygmomanometer has lost calibration. Warning of the absolute difference may be displayed by intermittent or flashing display about once per second . . . . Total display time may be 5-10 seconds
v) program end 82: the pressure measurement by the electronic sphygmomanometer blood is ended

Accordingly, while this invention has been described with reference to illustrative embodiments, this description is not intended to be construed in a limiting sense. Various modifications of the illustrative embodiments, as well as other embodiments of the invention, will be apparent to persons skilled in the art upon reference to this description. It is therefore contemplated that the appended claims will cover any such modifications or embodiments as fall within the true scope of the invention.

1. A method for automatic error detection in pressure measurement with a first and a second pressure sensing circuit and a MPU, said method comprising the steps of:
A) under the control of said MPU, keeping the first pressure sensing circuit normally on for measurement of pressure during normal use while keeping the second pressure sensing circuit normally off until being activated;
B) upon activation of said second pressure sensing circuit, acquiring pressure data from both pressure sensing circuits and comparing said pressure data to obtain pressure difference between said two pressure sensing circuits; and
C) upon detection of said pressure difference being over a given limit, sending out a pressure error warning.

2. A method as in claim 1, said method further comprising the step of keeping track of the usage time of said first pressure sensing circuit.

3. A method as in claim 2, said method further comprising the step of activating said second pressure sensing circuit whenever said usage time of said first pressure sensing circuit meets a give criterion.

4. A method as in claim 3, said given criterion is one of the following:
A) said usage time is a multiple of one day, one week, or one month; and
B) said usage time is a multiple of 2, 5, 10, 20 or 50 cycles of power on and off to said first pressure sensing circuit.

5. A method as in claim 1, wherein said method is used in an electronic sphygmomanometer of any type including automated, semi-automated and manually operated electronic sphygmomanometers.

6. An electronic sphygmomanometer for measurement of blood pressure of a subject, said sphygmomanometer comprising:
A) an inflatable cuff;
B) a first pressure sensing circuit that is normally power-on during said blood pressure measurement for measuring the pressure in said inflatable cuff;
C) a second pressure sensing circuit that is normally power-off during said blood pressure measurement until activated for measuring the same pressure in said inflatable cuff as the pressure that said first pressure sensing circuit measures; and
D) an MPU for acquiring the pressure data from said first and second pressure sensing circuits, for comparing said pressure data, and for sending out a pressure error warning whenever the difference between the pressure measured by said first and second pressure sensing circuits exceeds a give limit.

7. An electronic sphygmomanometer as in claim 6, wherein said MPU keeps track of the usage time of said first pressure sensing circuit.

8. An electronic sphygmomanometer as in claim 7, wherein said MPU activates said second pressure sensing circuit whenever said usage time of said first pressure sensing circuit meets a give criterion.

9. An electronic sphygmomanometer as in claim 8, wherein said given criterion is one of the following:
A) said usage time is a multiple of one day, one week, or one month; and
B) said usage time is a multiple of 2, 5, 10, 20 or 50 cycles of power on and off of said sphygmomanometer.

10. An electronic sphygmomanometer as in claim 8, wherein said MPU activates said second pressure sensing circuit by supplying power to it.
